(19) United States
(12) Patent Application Publication
(10) Pub. No.: US 2006/0287664 A1
(43) Pub. Date: Dec. 21, 2006

(54) Endcap for a fluid sampling device

(75) Inventors: Henry M. Grage Jr, Alpharetta, GA (US); Jack Griffis, Decatur, GA (US)

Correspondence Address: GARDNER GROFF SANTOS & GREENWALD, P.C., 2018 POWERS FERRY ROAD, SUITE 800, ATLANTA, GA, 30339, US

(73) Assignee: Facet Technologies, LLC., Atlanta, GA (US)

(21) Appl. No.: 10/570,225

(22) PCT Filed: Jul. 28, 2004
(86) PCT No.: PCT/US04/24281

§ 371 Date: Feb. 28, 2006

Related U.S. Application Data
(60) Provisional application No. 60/499,818, filed on Sep. 3, 2003.

(51) Int. Cl. A61B 17/32

(52) U.S. Cl. 606/181

(57) ABSTRACT

An endcap for a lancing device having an opening formed therein for passing a lancet tip, and a contact face surrounding the opening, with at least one spaced rib, dimple or projection thereon. In various embodiments, multiple ribs are spaced around the opening in a helical, circular or radial array. The contact face can be planar, concave or convex. The endcap increases the surface area in contact with the skin around the sampling site to enhance blood flow and prevent premature wound closure during sample collection.

### CROSS-REFERENCE TO RELATED APPLICATIONS

This application claims priority to U.S. Provisional patent application Ser. No. 60/499,818, filed Sep. 3, 2003, which is hereby incorporated herein by reference in its entirety for all purposes.

### TECHNICAL FIELD

The present invention relates generally to medical devices and procedures, and more particularly to lancing devices for sampling blood or other bodily fluids. The invention more particularly relates to an endcap for use in conjunction with a lancing device that allows for a collection of an increased volume of blood or bodily fluid to be obtained.

### BACKGROUND OF THE INVENTION

Many medical procedures require puncturing of the skin, and sometimes underlying tissues, of a human or animal subject. For example, a sharp lancet tip is commonly used to puncture the subject's skin for sampling of blood or other body fluid, as for example in blood glucose monitoring by diabetics. Generally, lancets are much thinner than traditional hypodermic syringe needles; and therefore result in less pain to the patient.

However, one common problem that has been found to exist with utilizing thin lancets is the closure of the puncture site before an adequate volume of blood has been collected. Premature closure of the puncture requires additional blood to be drawn from the patient, often in a separate and sometimes less desirable location, resulting in an increase in pain and stress. Costs also increase, as multiple lancets are required to acquire an adequate sample.

Attempts have been made to provide lancing devices that inhibit premature closure of the puncture site. Such past attempts have included the use of an outer ring positioned away from the puncture site to “milk” or apply pressure around the wound to express a larger sample and prevent closure of the skin. Although utilizing the aforementioned circular ring often allows a larger sample to be collected before closure of the puncture site, occasionally a sample size is still insufficient, and another lancing operation to collect blood or analytic fluid is required.

It is also desirable to minimize the pain sensed by the subject resulting from the lancing and sampling procedure. Pain causes many subjects to avoid sampling or to sample less frequently than is desirable, sometimes leading to adverse health consequences. Lancing is commonly carried out on the subject's fingers, typically on the tips of the fingers, where capillary density is relatively high near the skin surface, to produce good sample size. But because nerve concentration is also high in these regions, fingertip lancing often results in considerable pain for many subjects. Alternate lancing sites such as the forearm and other regions where nerve density is lower, however, often generate insufficient sample size with known lancing devices, commonly requiring multiple lancing procedures to be endured.

Thus it can be seen that needs exist for improved lancing devices and methods to provide a mechanism that allows for an increased volume of blood or other analytic fluid to be expressed and collected before the puncture site closes. Needs further exist for such a mechanism that is readily adaptable to current lancing devices and procedures.

### SUMMARY OF THE INVENTION

In example forms, the present invention is an endcap for a lancing device that allows for collection of an increased volume of blood or other body fluid from a lancing site on the skin of a human or animal subject. The present invention enhances sample collection from fingertip sampling sites, and is particularly well-suited for sampling from alternate sites such as the forearm and other sites with less pain sensitivity than the fingertips. In example embodiments, the device includes a series of raised ribs, which are compressed against the skin's surface around the lancing site to guide blood toward the wound for sample collection. In further embodiments, the invention is a method for increasing blood flow from a lancing site, the method including compressing a contact surface comprising a series of ribs against the skin around the lancing site, and optionally rotating or twisting the contact surface against the skin around the lancing site

In one aspect, the present invention is a lancing device for collecting a sample of body fluid from a sampling site on the skin of a subject. The lancing device preferably includes an outer body housing and a lancet, the lancet being movable between a first position within the outer body housing and a second position wherein at least a sharp tip portion of the lancet extends through an opening in the outer body housing. The lancing device preferably also includes a contact face surrounding the opening and at least one rib projecting from the contact face.

In another aspect, the invention is a lancing device for collecting a sample of body fluid from a sampling site on the skin of a subject. The lancing device preferably includes an outer body housing and a lancet, the lancet being movable between a first position within the outer body housing and a second position wherein at least a sharp tip portion of the lancet extends through an opening in the outer body housing. The lancing device preferably also includes a contact face surrounding the opening and at least one hemispherical dimple or projection on the contact face.

In another aspect, the invention is an endcap for a lancing device. The endcap preferably includes a first end for connection to the lancing device, and a second end defining an opening through which a sharp tip of a lancet can pass. The second end preferably also includes a contact face surrounding the opening and at least one rib projecting from the contact face.

In yet another aspect, the endcap of the present invention includes a plurality of ribs projecting from the contact face in a spaced array. In still another aspect, the ribs are spaced about the opening in a circular array.

In another aspect, the invention is an endcap for a lancing device. The endcap preferably includes a first end for connection to the lancing device, and a second end defining an opening through which a sharp tip of a lancet can pass. The second end preferably also includes a contact face surrounding the opening and at least one hemispherical dimple or projection on the contact face.

In another aspect, the invention is a method of sample collection with a lancing device. The method preferably includes the steps of lancing the skin of a subject at a lancing site, and compressing a ribbed contact face of the lancing device against the skin adjacent the lancing site. Optionally, the method further includes twisting the ribbed contact face in connection with the skin.

These and other aspects, features and advantages of the invention will be understood with reference to the drawing figures and detailed description herein, and will be realized by means of the various elements and combinations particularly pointed out in the appended claims. It is to be understood that both the foregoing general description and the following brief description of the drawings and detailed description of the invention are exemplary and explanatory of preferred embodiments of the invention, and are not restrictive of the invention, as claimed.

### BRIEF DESCRIPTION OF THE DRAWINGS

FIG. 1 shows a partial cross-sectional view of an endcap according to one example embodiment of the present invention.

FIG. 2 shows a perspective view of the endcap of FIG. 1.

FIG. 3 shows a front view of the endcap of FIG. 1.

FIG. 4 shows the concave profile of a contact face portion of an endcap according to another example embodiment of the present invention.

FIGS. 5-8 show detailed views of the ribbed contact face of endcaps according to several alternate embodiments of the present invention.

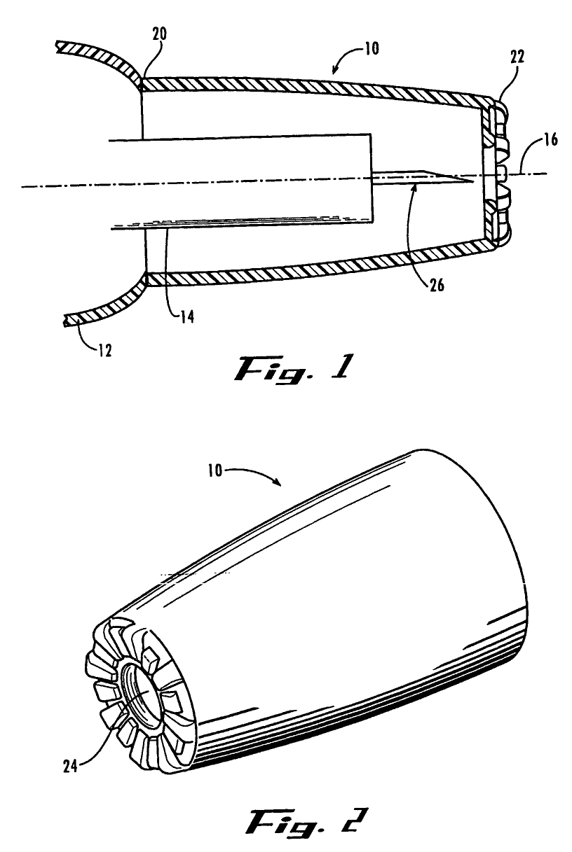

### DETAILED DESCRIPTION OF EXAMPLE EMBODIMENTS

The present invention may be understood more readily by reference to the following detailed description of the invention taken in connection with the accompanying drawing figures, which form a part of this disclosure. It is to be understood that this invention is not limited to the specific devices, methods, conditions or parameters described and/or shown herein, and that the terminology used herein is for the purpose of describing particular embodiments by way of example only and is not intended to be limiting of the claimed invention. Also, as used in the specification including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment.

With reference now to the drawing figures, an endcap 10 for a lancing device 12 housing a lancet 14 therein is shown by way of example embodiments of the present invention. The endcap 10 preferably is formed of a substantially rigid body having a longitudinal axis 16 extending from a first end 20 to a second end 22, wherein the first end 20 is sized and/or shaped to attach to the lancing device 12. The attachment of the endcap 10 to the lancing device 12 can be accomplished by way of a releasable or permanent coupling such as for example a threaded screw coupling, a socket, snap fittings, adhesive or welding, one or more screws or other connectors, or the like; or the endcap 10 can comprise an integral portion of the overall housing of the lancing device 12. In an example embodiment, at least a portion of the endcap 10 is formed of a transparent material to allow the patient and/or clinician to view the puncture site during lancing of the skin and collection of the bodily fluid, and thereby monitor the sample size.

The second end 22 of the endcap 10 defines an opening 24 through which a sharp lancet tip 25, such as a needle or blade tip, projects to lance the skin of the subject and form a wound at the sample site from which a sample of body fluid is expressed and collected. The opening 24 is preferably sized and shaped to allow passage of the tip 25 of the lancet 14 only, and to prevent the lancet 14 from escaping from the housing of the lancing device or getting stuck in the opening. The opening 24 is preferably also sized and shaped to prevent bodily fluids such as blood from contacting the endcap 10 during lancing and subsequent collection.

The second end 22 of the endcap 10 preferably comprises a contact face 26 surrounding or adjacent the opening 24. The contact face 26 is preferably concave, but in alternate embodiments is planar or convex. One or more, and preferably a plurality of discontinuous raised ribs 28 are preferably provided on the contact face 26. The ribs 28 increase the surface contact area between the skin and the contact face and assist in applying tension to the skin at the lancing site to enhance sample collection. The ribs 28 can be arranged in a regularly or irregularly spaced series, and optionally are oriented radially and lie in a generally ring-shaped pattern surrounding the opening 24. In one embodiment of the invention, the ribs 28 are of equal size and shape. In an alternate embodiment, the ribs are of differing lengths, widths and/or depths.

In one depicted embodiment, the ribs have a four-sided cross-section, as seen best in FIG. 3. In alternate embodiments, the ribs are circular, triangular or otherwise configured. Preferably, the ribs 28 are discontinuous raised features extending across part of the contact face 26, or more preferably across substantially the entire contact face 26 from the outer perimeter edge to the blood expression zone defined by the opening 24, to guide or direct available blood or other fluid in the tissue surrounding the lancing site toward the wound for sample collection, and/or to stimulate blood flow or prevent premature wound closure by application of tension and/or compression at and around the sampling site.

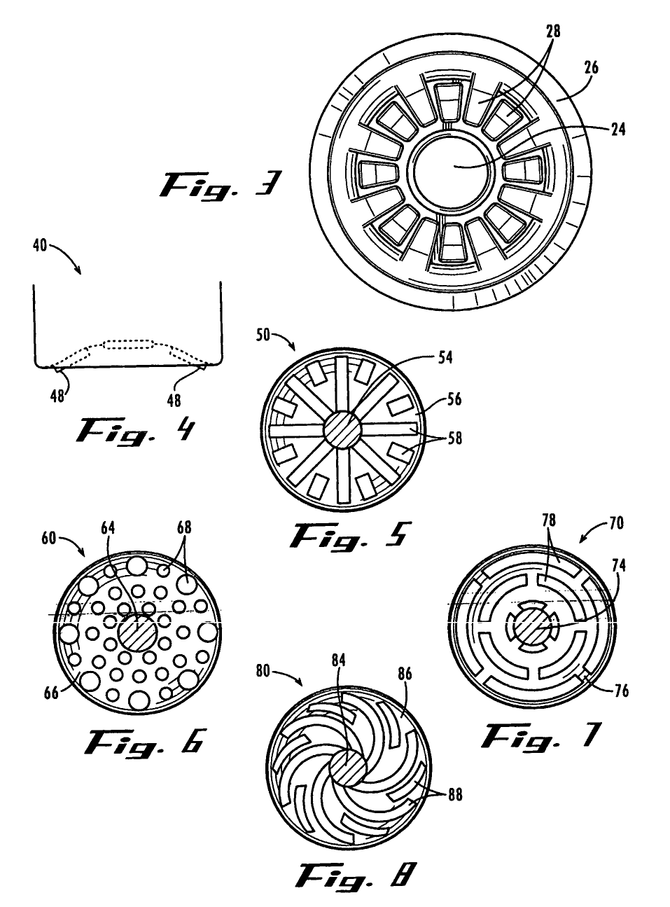

For example, FIGS. 4-8 show alternate embodiments of the present invention with various contact face and rib configurations. FIG. 4 depicts an endcap 40 having a concave contact face 46 from which one or more rib(s) or other surface features 48 project, for contact with the skin surface around the sampling site. The contact face 46 defines an opening 54 through which the sharp lancet tip 25, such as a needle or blade tip, projects to lance the skin of the subject.

FIG. 5 depicts an endcap 50 having a contact face 56 with a plurality of radial ribs 58 thereon positioned adjacent an opening 54. FIG. 6 depicts an endcap 60 having a contact face 66 with a plurality of hemispherical or otherwise-shaped recessed dimples 68 therein, surrounding an opening 64, in a pattern similar to dimples on a golf ball. In an alternate embodiment, the contact face comprises one or more raised hemispherical ribs projecting therefrom. FIG. 7 depicts an endcap 70 having a contact face 76 with a plurality of raised annular circumferential ribs 78 and interposed circumferential recesses thereon, surrounding an opening 74. FIG. 8 depicts an endcap 80 having a contact face 86 with a plurality of raised helical spiral ribs 88 and interposed helical recesses thereon surrounding an opening 84. The various rib embodiments can be applied on a contact face having a planar, concave, convex or irregular profile.

The present invention also includes a method of sample collection. In a preferred form, the method comprises compressing the ribbed contact face 26 against the skin adjacent the lancing site before and/or after lancing the skin at the lancing site. Optionally, the ribbed contact face 26 can be pumped to stimulate flow of the sampled fluid. Alternatively or additionally, the ribbed contact face is optionally twisted, rotated and/or rocked when in contact with the skin at the sampling site, to increase the skin tension at the sampling site and enhance sample collection. The provision of spaced ribs 28 on the contact surface 26 assists in gripping the skin surface on and between the ribs during this twisting operation.

While the invention has been described with reference to preferred and example embodiments, it will be understood by those skilled in the art that a variety of modifications, additions and deletions are within the scope of the invention, as defined by the following claims.

1. A lancing device for collecting a sample of body fluid from a sampling site on the skin of a subject, said lancing device comprising an outer body housing and a lancet, the lancet being movable between a first position within the outer body housing and a second position wherein at least a sharp tip portion of the lancet extends through an opening in the outer body housing, the lancing device further comprising a contact face surrounding the opening and a plurality of ribs projecting from the contact face in a ring-shaped pattern, each of said plurality of ribs having a first end adjacent the opening and extending lengthwise away from the opening and across the contact face.

2. The lancing device of claim 1, comprising a plurality of ribs projecting from the contact face in a regularly spaced array.

3. The lancing device of claim 2, wherein the ribs are spaced about the opening in a circular array.

4. The lancing device of claim 2, wherein the ribs are spaced about the opening in a radial array.

5. The lancing device of claim 2, wherein the ribs are spaced about the opening in a helical spiral array.

6. The lancing device of claim 2, wherein the ribs are spaced about the opening in an annular circumferential array.

7. The lancing device of claim 1, wherein the contact face is concave.

8. The lancing device of claim 1, wherein the contact face is convex.

9. A lancing device for collecting a sample of body fluid from a sampling site on the skin of a subject, said lancing device comprising an outer body housing and a lancet, the lancet being movable between a first position within the outer body housing and a second position wherein at least a sharp tip portion of the lancet extends through an opening in the outer body housing, the lancing device further comprising a contact face surrounding the opening and a plurality of recessed hemispherical dimples on the contact face.

10. An endcap for a lancing device, said endcap comprising a first end for connection to the lancing device, and a second end defining an opening through which a sharp tip of a lancet can pass, the second end further comprising a contact face surrounding the opening and a plurality of ribs projecting from the contact face in a ring-shaped pattern, each of said plurality of ribs having a first end adjacent the opening and extending lengthwise away from the opening and across the contact face.

11. The endcap of claim 10, comprising a plurality of ribs projecting from the contact face in a regularly spaced array.

12. The endcap of claim 11, wherein the ribs are spaced about the opening in a circular array.

13. The endcap of claim 11, wherein the ribs are spaced about the opening in a radial array.

14. The endcap of claim 11, wherein the ribs are spaced about the opening in a helical spiral array.

15. The endcap of claim 11, wherein the ribs are spaced about the opening in an annular circumferential array.

16. The endcap of claim 10, wherein the contact face is concave.

17. The endcap of claim 10, wherein the contact face is convex.

18. An endcap for a lancing device, said endcap comprising a first end for connection to the lancing device, and a second end defining an opening through which a sharp tip of a lancet can pass, the second end further comprising a contact face surrounding the opening and a plurality of recessed hemispherical dimples on the contact face.

19. A method collecting a sample of body fluid using a lancing device, said method comprising:
lancing the skin of a subject at a lancing site; and
compressing a ribbed contact face against the skin adjacent the lancing site, said ribbed contact face comprising a plurality of ribs extending lengthwise from an outer edge of the contact face inwardly toward an expression zone.

20. The method of claim 19, further comprising twisting the ribbed contact face in contact with the skin.
